(19) United States
(12) Patent Application Publication
(10) Pub. No.: US 2026/0199121 A1
(43) Pub. Date: Jul. 16, 2026

(54) Double Lumen Urine Drainage Tube with Integrated Pump

(71) Applicant: C. R. Bard, Inc., Franklin Lakes, NJ (US)

(72) Inventors: Esha Khurana, Chandigarh (IN); Nitin Kumar, Hamirpur (IN); Deepak Jagan, Bangalore (IN); Reema Chaudhary, Panchkula (IN)

(21) Appl. No.: 19/134,177

(22) PCT Filed: Nov. 30, 2022
(86) PCT No.: PCT/US2022/051431

§ 371 Date: May 29, 2025

(51) Int. Cl. A61F 5/451; A61M 1/00

(52) U.S. Cl. CPC A61F 5/451; A61M 1/60; A61M 1/80; A61M 1/84; A61M 2202/0496

(57) ABSTRACT

A urine drainage system includes a drainage tube and an outer tube extending between an interim container at a distal end and a connector at a proximal end. The drainage tube is also fluidly coupled with a urine collection container. The drainage tube is disposed with a lumen of an outer tube to define a closed annular chamber between the drainage tube and the outer tube. The system further includes an air pump in fluid communication with the annular chamber, where the air pump is configured to define a positive air pressure within the annular chamber. The drainage tube is configured to collapse as a result of the positive air pressure, and during use, collapsing the drainage tube displaces pooled urine within the drainage tube proximally toward the collection container. Different magnitudes of the positive pressure collapse different portions of the drainage tube.

### BACKGROUND

The draining of liquid (e.g., urine) may include the use of a liquid drainage system including a flexible drainage tube extending from a drainage catheter to a collection container. Typical catheters include indwelling catheters, Foley catheters, balloon catheters, peritoneal drainage catheters, or the like, and are configured to be inserted into an orifice within the body of a patient to drain a liquid therefrom. The flexibility of the drainage tube can form sections of positive incline, also termed “dependent loops,” where drainage liquid can accumulate. Liquid pooling within dependent loops can cause various complications. For example, urine pooling can be a source of catheter associated urinary tract infection (“CAUTI”) causing agents such as bacteria, microbes, and the like. Hospital Acquired Infections (“HAI”), such as CAUTI, are detrimental to the patient, and also incur extra costs in treating these additional complications. Embodiments disclosed herein are directed to clearing drainage liquid from dependent loops thereby, reducing patient risk.

### SUMMARY

Briefly summarized, systems and methods disclosed herein are directed to a urine drainage system configured to collect urine excreted from a patient. The system includes a drainage tube extending between an interim container at a distal end of the drainage tube and a connector at a proximal end of the drainage tube, where the drainage tube is in fluid communication with the interim container, and where the interim container is configured to receive urine from the patient via a urinary catheter. The drainage tube is also fluidly coupled with a collection container configured to collect the urine therein. The system further includes an outer tube extending between the interim container at a closed distal end of the outer tube and the connector at a closed proximal end of the outer tube. The drainage tube is disposed with a lumen of the outer tube to define an annular chamber surrounding the drainage tube. The system further includes an air pump in fluid communication with the annular chamber, where the air pump is configured to define a positive air pressure within the annular chamber. The drainage tube is configured to collapse as a result of the positive air pressure, and during use, collapsing the drainage tube displaces pooled urine within the drainage tube proximally toward the collection container.

In some embodiments of the system, the outer tube includes a flexible tubular wall such that outer tube is allowed to define a flattened shape in a free state. In some embodiments of the system, a stiffness of a tubular wall of the drainage tube is greater than a stiffness of the tubular wall of the outer tube.

In some embodiments of the system, a free length of the outer tube is greater than a free length of the drainage tube. In some embodiments of the system, in the absence of the positive air pressure, a distance between the interim container and the connector is less than the free length of the outer tube.

In some embodiments of the system, the positive air pressure defines a longitudinal tensile stress along the drainage tube.

In some embodiments of the system, the drainage tube is configured to (i) collapse at a first location along the drainage tube in response to a first positive air pressure and (ii) collapse at a second location along the drainage tube in response to a second positive air pressure, where the second positive air pressure is greater than the first positive air pressure, and where the second location is disposed proximal the first location.

In some embodiments of the system, the interim chamber includes air vent, configured to allow air to flow into and out of the interim chamber.

In some embodiments of the system, the drainage tube includes a one-way valve disposed in line with the drainage tube, where the one-way valve is configured to inhibit urine flow toward the interior container.

In some embodiments, the system further includes a console coupled with the air pump, where the console includes a processor and memory having logic stored thereon that, when executed by the processor, performs operations that include activating the air pump.

In some embodiments of the system, the operations further include repeatedly activating the air pump according to a defined time interval and in some embodiments of the system, the operations further include deactivating the air pump after a defined activation time period.

In some embodiments, the system further includes a pressure sensor in fluid communication with the annular chamber, where the pressure sensor configured to measure the positive air pressure within the annular chamber, and where the pressure sensor is coupled with the console, thereby enabling the logic to, at least one of, activate or deactivate the air pump in response to a pressure measurement acquired from the pressure sensor.

In some embodiments of the system, the operations further include comparing the pressure measurement with a high-pressure limit stored in memory, and as a result of the comparison, deactivating the pump when the pressure measurement exceeds the high-pressure limit.

In some embodiments of the system, the air pump is configured to relieve the positive pressure from the annular chamber when the air pump is deactivated.

In some embodiments of the system, the drainage tube, the outer tube, the interim container, and the connector are attached to each other to form a drainage tube assembly, and the air pump is separably attached to the drainage tube assembly.

Also disclosed herein is a method of draining urine from a patient. The method includes catheterizing the patient via a urinary catheter in fluid communication with a drainage tube of a drainage tube assembly, where the drainage tube is (i) coupled with the urinary catheter adjacent a distal end of the drainage tube and (ii) coupled with a collection container adjacent a proximal end of the drainage tube. The method further includes (i) establishing a flow of urine along the drainage tube from the urinary catheter to the collection container; (ii) applying a first positive pressure externally to the drainage tube along an entire length of the drainage tube to collapse a first portion of the drainage tube, where the first portion is disposed distal pooled urine within the drainage tube; and (iii) applying a second positive pressure externally to the drainage tube along the entire length of the drainage tube to collapse a second portion of the drainage tube, where the second portion is disposed proximal the first portion. In such embodiments, the second positive pressure is greater than the first positive pressure, and collapsing the second portion displaces the pooled urine proximally along the drainage tube.

In some embodiments of the method, collapsing the second portion causes a least a portion of the pooled urine to flow out of the drainage tube into the collection container.

In some embodiments of the method, the drainage tube assembly includes an outer tube extending along the entire length of the drainage tube, where the drainage tube is disposed with a lumen of the outer tube to define an annular chamber between the drainage tube and outer tube, and the annular chamber contains the first and second positive pressures.

In some embodiments, the method further includes removing the first and second positive pressures to allow the first and second portions to uncollapse.

In some embodiments, the method further includes applying a third positive pressure externally to the drainage tube along the entire length of the drainage tube to collapse a third portion of the drainage tube, where the third portion disposed proximal the second portion. In such embodiments, the third positive pressure is greater than the second positive pressure, and collapsing the third portion displaces the pooled urine proximally along the drainage tube such that at least another portion of the pooled urine flows out of the drainage tube into the collection container.

In some embodiments of the method, the annular chamber is fluidly coupled with an air pump, the air pump configured to define the first positive pressure when the air pump is activated.

In some embodiments of the method, the drainage tube assembly includes an interim chamber coupled in line between the urinary catheter and the drainage tube, and the method further includes (i) visually detecting a collection of urine within the interim chamber and (ii) activating the air pump in response to visually detecting the collection of urine.

In some embodiments, removing the first positive pressure includes deactivating the air pump.

Also disclosed herein is a method of purging pooled urine from a urine drainage tube. According to some embodiments, the method includes applying a first positive pressure externally to the drainage tube along an entire length of the drainage tube to collapse a first portion of the drainage tube, where the first portion disposed distal pooled urine within the drainage tube. The method further includes applying a second positive pressure externally to the drainage tube along the entire length of the drainage tube to collapse a second portion of the drainage tube, where the second portion disposed proximal the first portion. In such embodiments, the drainage tube is (i) coupled with a urinary catheter adjacent a distal end of the drainage tube and (ii) coupled with a collection container adjacent a proximal end of the drainage tube, the second positive pressure is greater than the first positive pressure, and collapsing the second portion displaces the pooled urine proximally along the drainage tube.

In some embodiments of the method, collapsing the second portion causes a least a portion of the pooled urine to flow out of the drainage tube into the collection container.

In some embodiments of the method, the drainage tube assembly includes an outer tube extending along the entire length of the drainage tube. In such embodiments, the drainage tube is disposed with a lumen of the outer tube to define an annular chamber between the drainage tube and outer tube, and the annular chamber contains the first and second positive pressures.

In some embodiments, the method further includes removing the first and second positive pressures to allow the first and second portions to uncollapse.

In some embodiments, the method further includes applying a third positive pressure externally to the drainage tube along the entire length of the drainage tube to collapse a third portion of the drainage tube, where the third portion is disposed proximal the second portion. In such embodiments, the third positive pressure is greater than the second positive pressure, and collapsing the third portion displaces the pooled urine proximally along the drainage tube such that at least another portion of the pooled urine flows out of the drainage tube into the collection container.

In some embodiments of the method, the annular chamber is fluidly coupled with an air pump, and the air pump is configured to define the first positive pressure when the air pump is activated.

In some embodiments of the method, the drainage tube assembly includes an interim chamber coupled in line between the urinary catheter and the drainage tube, and the method further includes (i) visually detecting a collection of urine within the interim chamber and (ii) activating the air pump in response to visually detecting the collection of urine.

In some embodiments, removing the first positive pressure includes deactivating the air pump.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows an exemplary urine collection system;

FIG. 2 shows a urine collection system including an air pump coupled with an annular chamber extending along a drainage tube, in accordance with some embodiments disclosed herein;

FIG. 3A illustrates a side view of an outer tube of the system of FIG. 2, in accordance with some embodiments disclosed herein;

FIG. 3B illustrates a side view of a drainage tube of the system of FIG. 2, in accordance with some embodiments disclosed herein;

FIG. 3C illustrates a side view of a drainage tube assembly of the system of FIG. 2 in a free state, in accordance with some embodiments disclosed herein;

FIGS. 4A-4E illustrate side views of the drainage tube assembly according to various states of a urine purging process, in accordance with some embodiments disclosed herein.

FIG. 5 is a flow chart of an exemplary method of draining urine from a patient, in accordance with some embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The phrases “connected to,” “coupled to/with,” and “in communication with” refer to any form of interaction between two or more entities, including but not limited to mechanical, electrical, magnetic, electromagnetic, fluid, and thermal interaction. Two components may be coupled to each other even though they are not in direct contact with each other. For example, two components may be coupled to each other through an intermediate component.

The terms “proximal” and “distal” refer to opposite ends of a medical device, including the devices disclosed herein. As used herein, the proximal portion of a medical device is the portion nearest a practitioner during use, while the distal portion is the portion at the opposite end. For example, the proximal end of a drainage tube is defined as the end closest to the practitioner during utilization of the drainage tube. The distal end is the end opposite the proximal end, along the longitudinal direction of the drainage tube.

Unless defined otherwise, all technical and scientific terms used herein have the same meaning as commonly understood by those of ordinary skill in the art.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified. A subset of the steps or actions for one method may define a separate method.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially straight” is recited with respect to a feature, it is understood that in further embodiments, the feature can have a precisely straight configuration.

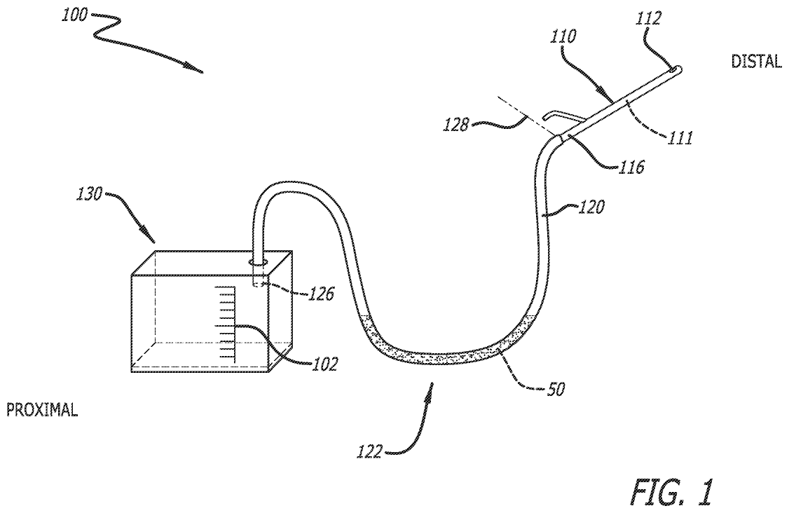

FIG. 1 shows an exemplary fluid collection system 100, which generally includes a catheter 110 defining a catheter lumen 111, a drainage tube (“tube”) 120, and a collection container (“container”) 130. Exemplary catheters 110 include indwelling catheters, Foley catheters, balloon catheters, peritoneal drainage catheters, or the like, and are configured to be inserted into an orifice within the body of a patient to drain a fluid therefrom. In an embodiment, the catheter 110 can be inserted through the urethra and into a bladder of a patient. The catheter 110 includes an eyelet 112 that provides fluid communication with a lumen of the catheter 110, and is configured to drain a fluid, e.g. urine.

The drainage tube 120 extends from a distal end 128 to a proximal end 126 to define an axial length, and defines a lumen. The distal end 128 of the drainage tube 120 is configured to engage a proximal 116 end of the catheter 110. The drainage tube 120 provides fluid communication between the catheter lumen 111 and the container 130. The drainage tube 120 can be formed of rubber, plastic, polymer, silicone, or similar suitably compliant material. The collection container 130 can include a rigid container, a flexible collection bag, or similar suitable container for receiving a fluid, e.g., urine drained from the catheter 110. In an embodiment, the container 130 includes graduated markings 102 for measuring a fluid disposed therein. As shown in FIG. 1, dependent loops 122 can form when slack portions of the drainage tube 120 create a positive incline relative to the direction of fluid flow. This can lead to fluid pooling of urine 50 within the drainage tube 120 which, in some instances, can be detrimental to the patient.

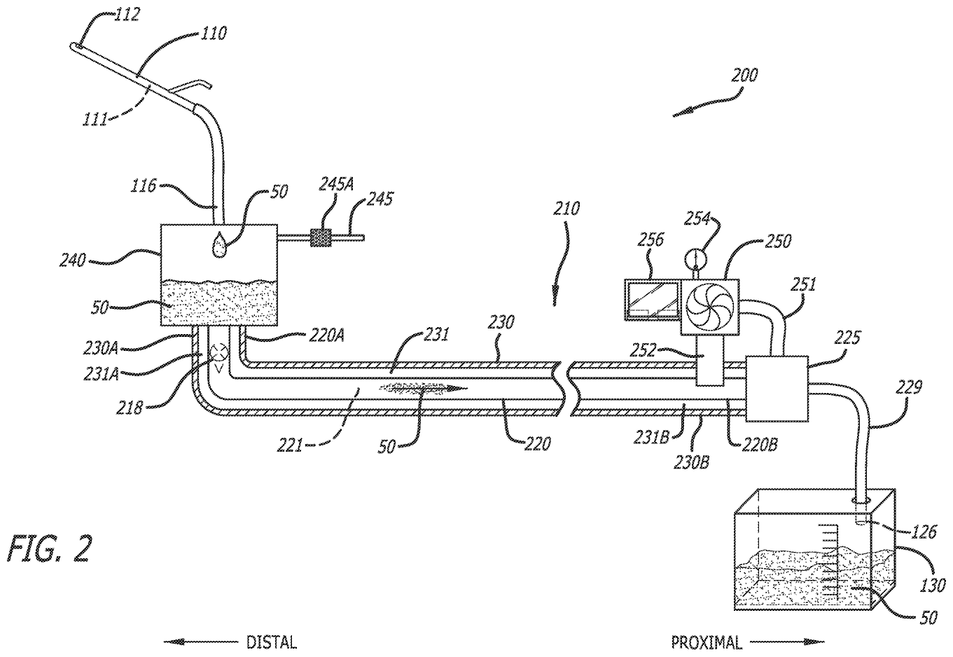

FIG. 2 shows a urine collection system (“system”) 200 configured to perform a urine purging process to remove pooled urine from the drainage tube. The system 200 includes the drainage tube 220 extending between an interim urine container 240 at a distal end 220A of the drainage tube 220 and connector 225 at a proximal end 220B. The drainage tube 220 may in certain respects resemble the components and functionality of the drainage tube 120 of FIG. 1. In the illustrated embodiment, system 200 includes a second drainage tube 229 extending between the connector 225 and collection container 130. In some embodiments, the second drainage tube 229 may be an extension of the drainage tube 220, i.e., the drainage tube 220 may pass through the connector 225 and couple directly with the collection container 130. During use, the urine 50 may flow from the catheter lumen 111 through a drainage tube lumen 221 of the drainage tube 220, including the second drainage tube 229, into the container 130. In some embodiments, the second drainage tube 229 may be omitted such that the connector 225 is directly coupled with collection container 130.

In the illustrated embodiment, the catheter 110 may be coupled with the interim container 240 so that urine 50 flowing through the catheter 111 lumen flows into the interim container 240. The interim container 240 can include a rigid container, a flexible collection bag, or similar suitable container for receiving the urine 50. In some embodiments, the interim container 240 may temporally collect a portion urine 50 excreted from the patient. For example, in some instances, the urine flow from the catheter 110 into the interim container 240 may exceed the urine flow out of the interim container 240 for a period of the time. By way of one exemplary instance, urine 50 pooled within the drainage tube 220 may reduce, inhibit, or prevent flow of urine 50 along the drainage tube 220, in which instance, the urine 50 may collect within the interim container 240. In some embodiments, the interim container 240 may be transparent or translucent such that the clinician may visually observe urine 50 collecting within the interim container 240, thereby determining that the urine 50 has pooled within the drainage tube 220.

In some embodiments, the interim container 240 may define a pressure buffer for the urinary catheter 110. In other words, the interim container 240 may minimize a pressure increase or a pressure decrease within the urinary catheter 110 during use, such as when the flow of urine along the drainage tube 220 is interrupted, for example. The pressure buffer may also reduce discomfort to the patient and/or reduce the risk of injury to the patient when the flow of urine along the drainage tube 220 is interrupted and/or when pooled urine is purged from the drainage tube 220.

In some embodiments, the interim container 240 may include an air vent 245. The air vent 245 may be configured to isolate the catheter 110 from pressure within the drainage tube 120. The air vent 245 may be configured to prevent urine 50 from exiting the interim container 240 via the air vent 245. In some embodiments, the air vent 245 may include a sterilizing filter 245A (e.g., a filter having a pore size less than 0.2 μm) extending across a lumen of the air vent 245. In some embodiments, the sterilizing filter 245A may be hydrophobic, thereby preventing passage of urine therethrough.

In an embodiment, the system 200 can further include a one-way valve 218, disposed at the distal end 220A of the drainage tube 220. The one-way valve 218 is designed to prevent retrograde flow of urine distally along the drainage tube 220 toward the interim container 240. In an embodiment, one or more of the catheter 110, valve 218, the drainage tube 220, interim container 240, the connector 225, the second drainage tube 229, or the collection container 130, may be connected to an adjacent component using a Luer lock, spin nut, or similar suitable fluid connection.

The system 200 further includes an outer tube 230 extending along the drainage tube 220. In the illustrated embodiment, the outer tube 230 extends between the interim urine container 240 at a distal end 230A of the outer tube 230 and the connector 225 at a proximal end 230B. The drainage tube 220 is disposed within a lumen of the outer tube 230 to define an annular chamber 231 surrounding the exterior of the drainage tube 220. The annular chamber 231 extends between a closed distal end 231A and a closed proximal end 231B. The outer tube 230 can be formed of rubber, plastic, polymer, silicone, or any suitable medical tubing material. In some embodiments, the interim container drainage tube 220, the outer tube 230, the interim container 240 and the connector 225 may define a drainage tube assembly 210.

The system 200 further includes an air pump 250 fluidly coupled with the annular chamber 231 such that activating the air pump 250 pressurizes the annular chamber 231. In some embodiments, deactivating the air pump 250 may relieve pressure from the annular chamber 231. The pressure within the annular chamber 231 exerts crushing or occluding force on the exterior of the drainage tube 220 as further described below. In the illustrated embodiment, an air hose 251 couples the air pump 250 with the annular chamber 231. In some embodiments, the closed proximal end 231B of the annular chamber 231 is defined by the connector 225 and the air hose 251 is coupled with the connector 225. In the illustrated embodiment, the air pump 250 is attached to the outer tube 230 or the connector 225 via an attachment device 252, such as a clip, a clamp, a strap, or any other suitable attachment device that enables the air pump to be attached to and detached from the outer tube 230 or the connector 225. In some embodiments of use, the clinician may manually activate and/or deactivate the air pump 250.

The air pump 250, or the system 200 generally, may optionally include pressure sensor 254 in fluid communication with the air hose 251, where the pressure sensor 254 is configured to measure or otherwise detect a pressure within the air hose 254 and/or the annular chamber 231.

The air pump 250 may optionally include a console 256 having a processor and logic stored in memory (e.g., a non-transitory computer-readable medium). The logic when executed by the processor may in some instances govern the operation the air pump 250. For example, the logic may activate and/or deactivate the air pump 250 according to defined time periods. By way of one example, the logic may activate the air pump 250 according to defined time interval, such as every 10 minutes, for example. Similarly, the logic may deactivate the air pump 250 after a defined activation time period, such as after 10 seconds, for example.

In some embodiments, the console 256 may be coupled with the pressure sensor 254, and the logic may be configured to activate and/or deactivate the air pump 250 based on a pressure within the annular chamber 231 as measured by the pressure sensor 254. For example, the logic may activate the air pump 250 until the pressure within the annular chamber 231 achieves the first positive pressure 401 to remove a kink or inhibit a kink from forming along the drainage tube 220. Similarly, the logic may modulate the air pump 250 to maintain a pressure with the annular chamber 231 at about the first positive pressure 401. According to another example, the logic may compare the pressure within the annular chamber 231 as measured by the pressure sensor 254 with a high-pressure limit stored in the memory. As a result of the comparison, the logic may deactivate the air pump 250 when the pressure exceeds the high-pressure limit.

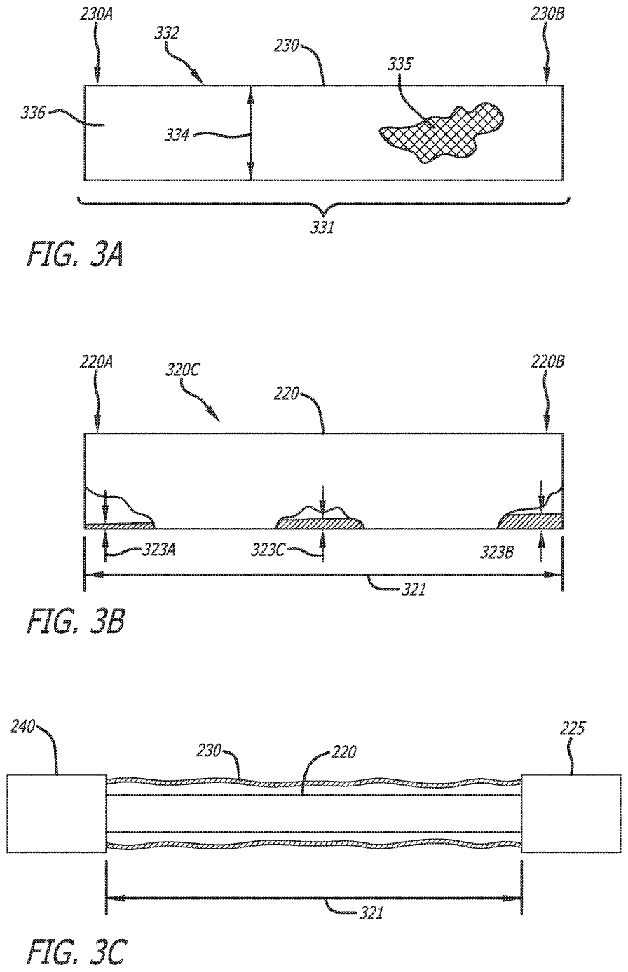

FIG. 3A is a detailed illustration of the outer tube 230, according to some embodiments. The outer tube 230 may generally defines a free length 331 extending between the distal end 230A and the proximal end 230B. In some embodiments, a tubular wall 332 of the outer tube 230 may be significantly flexible in bending such that in the free state (i.e., generally free of stress) the outer tube 230 may self-deform away from a cylindrical shape. In some embodiments, the outer tube 230 may generally form a flattened shape in the free state. The flattened shape in the free state may be advantageous during storage and shipping of the drainage tube assembly 210 because the outer tube 230 may fit in a smaller package than otherwise.

The tubular wall 332 of the outer tube 230 may be relatively stiff in tension. In other words, when the outer tube 230 defines a cylindrical shape, a diameter 334 of the outer tube 230 may remain substantially constant when a pressure with a lumen 336 of the outer tube 230 is increased. In some embodiments, the tubular wall 332 may include tensile fibers 335, such as glass or carbon fibers, for example, embedded therein to add tensile strength to the tubular wall 332.

FIG. 3B is a detailed illustration of the drainage tube 220, according to some embodiments. The drainage tube 220 may generally defines a free length 321 extending between the distal end 220A and the proximal end 220B. The drainage tube 220 is also configured such that the drainage tube lumen 221 is open in the free state. In some embodiments, the drainage tube lumen 221 may define a circular cross section in the free state. In other embodiments, the drainage tube lumen 221 may define an oval or some other cross section in the free state.

The drainage tube 220 is generally configured to flatten (i.e., cause the drainage tube lumen 221 to substantially close or occlude) when exposed to an external pressure. In some embodiments, one portion of the drainage tube 220 may be configured to collapse or flatten more easily (i.e., in response to less external pressure) than another portion of the drainage tube 220 when exposed to the external pressure.

In some embodiments, the drainage tube 220 may be include a non-constant tubular wall thickness. For example, a tubular wall thickness adjacent the distal end 220A may be different than a tubular wall thickness adjacent the proximal end 220B. In the illustrated exemplary embodiment, the drainage tube 220 includes a distal wall thickness 323A adjacent the distal end 220A and a proximal wall thickness 323B adjacent the proximal end 220A, where the distal wall thickness 323A is less than the proximal wall thickness 323B. As such, the drainage tube 220 may be configured to flatten more easily adjacent the distal end 220A than adjacent the proximal end 220B when exposed to the external pressure. In some embodiments, the drainage tube 220 may include central wall thickness 323C adjacent a central portion 320C of the drainage tube 220, where the central wall thickness 323C is greater than the distal wall thickness 323A and less than the proximal wall thickness 323B. In some embodiments, the tubular wall thickness may gradually transition between the distal wall thickness 323A and the proximal wall thickness 323B.

FIG. 3C is a detailed illustration of the drainage tube assembly 210 in a free state, according to some embodiments. In some embodiments, the free length 321 of the drainage tube 220 may define the distance between the interim container 240 and the connector 225 in the free state. In some embodiments, the free length 331 of the outer tube 230 may be longer than the free length 321 of the drainage tube 220. As such, when the drainage tube assembly 210 is disposed in the free state, the outer tube 230 may be partially longitudinally collapsed (i.e., shortened). In other embodiments, the free length 321 of the drainage tube 220 and the free length 331 of the outer tube 230 may be substantially the same.

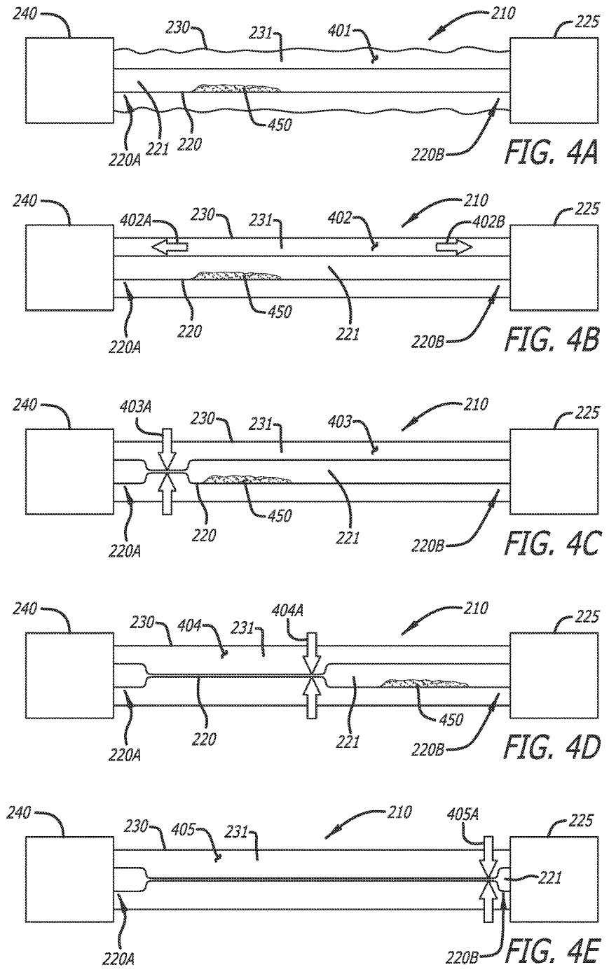

FIGS. 4A-4E illustrate the drainage tube assembly 210 in sequential states of the urine purging process. Each of the FIGS. 4A-4E show the drainage tube 220 and the outer tube 230 extending between the interim container 240 and the connector 225. The various pressures within the annular chamber 231 are defined by the air pump 250 (see FIG. 2). In the illustrated embodiment, the free length 331 of the outer tube 230 is longer than the free length 321 of the drainage tube 220.

FIG. 4A illustrates the drainage tube assembly 210 in a free state, i.e., having essentially zero pressure within the annular chamber 231. The outer tube 231 is shown as partially collapsed (shortened). In some instances, the urine may flow freely along the drainage tube 220 without pooling. In such instances, the purging process may be unnecessary. The state depicted in FIG. 4A may be consistent with such instances. In the illustrated case, pooled urine 450 is present within the drainage tube, such as illustrated in FIG. 4A.

The drainage tube 220 extends between the interim container 240 and the connected 225 in the free state. The outer tube 230 is shown in a partially longitudinally collapsed or shortened state. Although not shown, all or portions of the outer tube 230 may also be disposed in a flattened shape. A first air pressure 401 is resides within the annular chamber 231. The first air pressure 401 is essentially zero (i.e., zero gauge pressure) consistent with the air pump 250 disposed in a deactivated state.

FIG. 4B illustrates the drainage tube assembly 210 having a second positive pressure 402 residing within the annular chamber 231, where the second positive pressure 402 is greater than the first positive pressure 401 of FIG. 4A. As a result of the second positive pressure 402 residing within the annular chamber 231, the outer tube 230 is inflated such that no portion of the outer tube is disposed in a flattened shape. The second positive pressure 402 exerts opposing longitudinal forces 402A, 402B on the interim container 240 and the connector 225, respectively. In some embodiments, the longitudinal forces 402A, 402B may induce a longitudinal stress along the drainage tube 225. Although not specifically depicted, the outer tube 230 may remain disposed in the partially longitudinally collapsed or shortened state, i.e., the instant length of the outer tube 230 may less than the free length 331 (see FIG. 3A) of the outer tube 230.

Although not specifically depicted, the longitudinal stress along the drainage tube 220 may be configured to remove a kink from the drainage tube 220 or inhibit a kink from forming along the drainage tube 220. In other words, the drainage tube assembly 210 may be configured such that the air pressure within the annual chamber 231 causes the drainage tube 220 to straighten and/or longitudinally stretch, thereby removing a kink or inhibiting the formation of a kink along the drainage tube 220.

FIG. 4C illustrates the drainage tube assembly 210 having a third positive pressure 403 residing within the annular chamber 231, where the third positive pressure 403 is greater than the second positive pressure 402 of FIG. 4B. The third positive pressure 403 defines a collapsing force 403A on the drainage tube 220, where the collapsing force 403A is sufficient collapse or flatten the drainage tube 220 adjacent the distal end 220A. As discussed above in relation to the FIG. 3B, the drainage tube 220 may more easily collapse or flatten in response to an external pressure than other portions of the drainage tube 220. In the illustrated embodiment, the drainage tube 220 may more easily collapse or flatten adjacent the distal end 220A. The collapsed state the drainage tube 220 may inhibit flow of urine toward the interim container 240.

FIG. 4D illustrates the drainage tube assembly 210 having a forth pressure 404 residing within the annular chamber 231, where the fourth pressure 404 is greater than the third positive pressure 403 of FIG. 4C. The fourth pressure 404 defines a collapsing force 404A on the drainage tube 220, where the collapsing force 404A is sufficient collapse or flatten the drainage tube 220 along a portion of the drainage tube 220 spaced away from the distal end 220A. The collapsing state the drainage tube 220, resulting from the fourth pressure 404, may displace the pooled urine 450 proximally toward the connector 225.

FIG. 4E illustrates the drainage tube assembly 210 having a fifth pressure 405 residing within the annular chamber 231, where the fifth pressure 405 is greater than the fourth pressure 404 of FIG. 4D. The fifth pressure 405 defines a collapsing force 405A on the drainage tube 220, where the collapsing force 405A is sufficient to collapse or flatten the drainage tube 220 along a portion of the drainage tube 220 spaced further away from the distal end 220A, such as adjacent the proximal end 220B. The collapsing state the drainage tube 220, resulting from the fifth pressure 405, may substantially displace the pooled urine 450 out of the drainage tube 220 and into the collection container 130.

In some embodiments, the purging process may include activating the air pump 250. A result of the activation, the pressure within annular chamber 231 may increase from the first positive pressure 401 to the fifth pressure 405 as the air pump 250 continues to operate. In response to the increasing pressure, the drainage tube 220 may first collapse adjacent the distal end 220A after which, the drainage tube 220 may continue to collapse further proximally along the length of the drainage tube 220 toward the proximal end 220B, thereby causing the pooled urine 450 to displace proximally along the drainage tube 220 to remove the pooled urine 450 from the drainage tube 220.

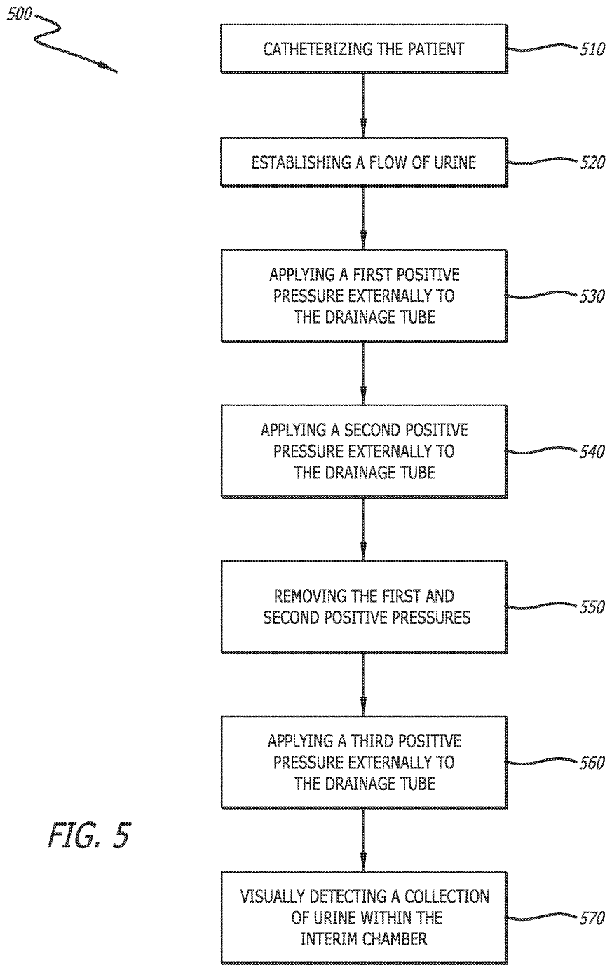

FIG. 5 is a flow chart of an exemplary method of draining urine from a patient. The method 500 may include all or any subset of the following steps, action, or processes. The method 500 may include catheterizing the patient (block 510), i.e., inserting the urinary catheter 110 within the patient. In some embodiments, the urinary catheter may be in fluid communication with the drainage tube of a drainage tube assembly.

The method 500 may further include establishing a flow of urine along the drainage tube (block 520), where the urine flows proximally long the drainage tube from the urinary catheter to the collection container. In some embodiments, the urine flows through the interim container.

The method 500 may further include applying a first positive pressure externally to the drainage tube (block 530) where the first positive pressure is applied along an entire length of the drainage tube. The first positive pressure causes a first portion of the drainage tube to collapse. In some instances, the urine may be pooled in a dependent loop and the first portion of the drainage tube may be disposed distal the pooled urine. In some embodiments, the first portion in the collapsed state may substantially occlude the drainage tube so that urine flow along the first portion is inhibited.

The method 500 may further include applying a second positive pressure externally to the drainage tube (block 540), where the second positive pressure is applied along an entire length of the drainage tube. The second positive pressure causes a second portion of the drainage tube to collapse, where the second portion is disposed proximal the first portion, and where the second positive pressure is greater than the first positive pressure. Collapsing the second portion displaces the pooled urine proximally along the drainage tube. In some embodiments, collapsing the second portion causes a least a portion of the pooled urine to flow out of the drainage tube into the collection container.

In some embodiments, the drainage tube assembly includes an outer tube extending along the entire length of the drainage tube, and the drainage tube is disposed with a lumen of the outer tube to define an annular chamber between the drainage tube and outer tube, where the annular chamber contains the first and second positive pressures.

The method 500 may further include removing the first and second positive pressures to allow the first and second portions to uncollapse (block 550). In some embodiments, removing the first positive pressure includes deactivating the air pump.

The method 500 may further include applying a third positive pressure externally to the drainage tube (block 560) along the entire length of the drainage tube to collapse a third portion of the drainage tube, where the third portion is disposed proximal the second portion, and where the third positive pressure is greater than the second positive pressure. In some embodiments, collapsing the third portion displaces the pooled urine proximally along the drainage tube such that at least another portion of the pooled urine flows out of the drainage tube into the collection container.

In some embodiments, the annular chamber is fluidly coupled with an air pump, where the air pump configured to define the first positive pressure when the air pump is activated. Similarly, the air pump may be configured to define the second and third positive pressures when the air pump is activated.

In some embodiments, the drainage tube assembly includes an interim chamber coupled in line between the urinary catheter and the drainage tube, the method 500 may further include visually detecting a collection of urine within the interim chamber (block 570) and activating the air pump in response to visually detecting the collection of urine.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A urine drainage system, comprising:
a drainage tube extending between an interim container at a distal end of the drainage tube and a connector at a proximal end of the drainage tube, the drainage tube in fluid communication with the interim container, wherein the interim container is configured to receive urine from a patient via a urinary catheter;
an outer tube extending between the interim container at a closed distal end of the outer tube and the connector at a closed proximal end of the outer tube, the drainage tube disposed with a lumen of the outer tube to define an annular chamber surrounding the drainage tube; and
an air pump in fluid communication with the annular chamber, the air pump configured to define a positive air pressure within the annular chamber;
wherein:
the drainage tube is fluidly coupled with a collection container,
the drainage tube is configured to collapse as a result of the positive air pressure, and
during use, collapsing the drainage tube displaces pooled urine within the drainage tube proximally toward the collection container.

2. The system according to claim 1, wherein the outer tube includes a flexible tubular wall such that the outer tube is allowed to define a flattened shape in a free state.

3. The system according to claim 2, wherein a stiffness of a tubular wall of the drainage tube is greater than a stiffness of the tubular wall of the outer tube.

4. The system according to claim 1, wherein a free length of the outer tube is greater than a free length of the drainage tube.

5. The system according to claim 4, wherein in the absence of the positive air pressure, a distance between the interim container and the connector is less than the free length of the outer tube.

6. The system according to claim 5, wherein the positive air pressure defines a longitudinal tensile stress along the drainage tube.

7. The system according to claim 1, wherein:
the drainage tube is configured to:
collapse at a first location along the drainage tube in response to a first positive air pressure, and
collapse at a second location along the drainage tube in response to a second positive air pressure,
the second positive air pressure is greater than the first positive air pressure, and
the second location is disposed proximal the first location.

8. The system according to claim 1, wherein the interim chamber includes air vent configured to allow air to flow into and out of the interim chamber.

9. The system according to claim 1, wherein the drainage tube includes a one-way valve disposed in line with the drainage tube, the one-way valve configured to inhibit urine flow toward the interim container.

10. The system according to claim 1, further comprising a console coupled with the air pump, the console including a processor and memory having logic stored thereon that, when executed by the processor, performs operations that include activating the air pump.

11. The system according to claim 10, wherein the operations further include repeatedly activating the air pump according to a defined time interval.

12. The system according to claim 10, wherein the operations further include deactivating the air pump after a defined activation time period.

13. The system according to claim 10, further comprising a pressure sensor in fluid communication with the annular chamber, the pressure sensor configured to measure the positive air pressure within the annular chamber, wherein the pressure sensor is coupled with the console, thereby enabling the logic to, at least one of, activate or deactivate the air pump in response to a pressure measurement acquired from the pressure sensor.

14. The system according to claim 13, wherein the operations further include:
comparing the pressure measurement with a high-pressure limit stored in the memory; and
as a result of the comparison, deactivating the pump when the pressure measurement exceeds the high-pressure limit.

15. The system according to claim 1, wherein the air pump is configured to relieve the positive pressure from the annular chamber when the air pump is deactivated.

16. The system according to claim 1, wherein:
the drainage tube, the outer tube, the interim container, and the connector are attached to each other to form a drainage tube assembly, and
the air pump is attached to the drainage tube assembly.

17. A method of draining urine from a patient, comprising:
catheterizing the patient via a urinary catheter in fluid communication with a drainage tube of a drainage tube assembly, the drainage tube (i) coupled with the urinary catheter adjacent a distal end of the drainage tube and (ii) coupled with a collection container adjacent a proximal end of the drainage tube;
establishing a flow of urine along the drainage tube from the urinary catheter to the collection container;
applying a first positive pressure externally to the drainage tube along an entire length of the drainage tube to collapse a first portion of the drainage tube, the first portion disposed distal pooled urine within the drainage tube; and
applying a second positive pressure externally to the drainage tube along the entire length of the drainage tube to collapse a second portion of the drainage tube, the second portion disposed proximal the first portion,
wherein:
the second positive pressure is greater than the first positive pressure, and
collapsing the second portion displaces the pooled urine proximally along the drainage tube.

18. The method according to claim 17, wherein collapsing the second portion causes a least a portion of the pooled urine to flow out of the drainage tube into the collection container.

19. The method according to claim 17, wherein:
the drainage tube assembly includes an outer tube extending along the entire length of the drainage tube,
the drainage tube is disposed with a lumen of the outer tube to define an annular chamber between the drainage tube and outer tube, and
the annular chamber contains the first and second positive pressures.

20. The method according to claim 17, further comprising removing the first and second positive pressures to allow the first and second portions to uncollapse.

21. The method according to claim 17, further comprising:
applying a third positive pressure externally to the drainage tube along the entire length of the drainage tube to collapse a third portion of the drainage tube, the third portion disposed proximal the second portion,
wherein:
the third positive pressure is greater than the second positive pressure, and
collapsing the third portion displaces the pooled urine proximally along the drainage tube such that at least a portion of the pooled urine flows out of the drainage tube into the collection container.

22. The method according to claim 17, wherein the annular chamber is fluidly coupled with an air pump, the air pump configured to define the first positive pressure when the air pump is activated.

23. The method according to claim 17, wherein the drainage tube assembly includes an interim chamber coupled in line between the urinary catheter and the drainage tube, the method further comprising:
visually detecting a collection of urine within the interim chamber; and
activating the air pump in response to visually detecting the collection of urine.

24. The method according to claim 20, wherein removing the first positive pressure includes deactivating the air pump.

25-32. (canceled)
